(19) United States
(12) Patent Application Publication
(10) Pub. No.: US 2013/0084226 A1
(43) Pub. Date: Apr. 4, 2013

(54) SENSING TEST BLOCK WITH RAPID CONDUCTIVE REACTION EFFECT

(71) Applicants: Tong-Yuh HUANG, Hsin-chu County (TW); Hui-Fang Wang, Hsin-chu County (TW)

(72) Inventors: Tong-Yuh HUANG, Hsin-chu County (TW); Hui-Fang Wang, Hsin-chu County (TW)

(73) Assignee: SAND COUNTY BIOTECHNOLOGY, INC., Hu-ko (TW)

(21) Appl. No.: 13/250,059

(22) Filed: Sep. 30, 2011

(51) Int. Cl. G01N 33/48

(52) U.S. Cl. 422/400; 422/68.1; 422/82.01

(57) ABSTRACT

A sensing test block with rapid conductive reaction effect includes a test block body, reaction detector, reaction space and chemical reaction layer. A porous separation and filtering layer and a capillary guiding and diffusion portion are set onto the chemical reaction layer. The porous separation and filtering layer could separate the blood corpuscle in the test blood sample due to its smaller aperture, and the porous separation and filtering layer is provided with a guiding portion that is mated with the specimen inlet. The capillary guiding and diffusion portion is provided with a specimen guiding portion that is mated with the specimen inlet. A venting portion is set onto the capillary guiding and diffusion portion, and located correspondingly to the external side of the porous separation and filtering layer, making the venting portion farther from the specimen inlet than the porous separation and filtering layer.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC Not applicable.

### BACKGROUND OF THE INVENTION

1. Field of the Invention

The present invention relates generally to an electrochemical sensing test block, and more particularly to an innovative one which allows a capillary guiding & diffusion portion to be set over a porous separation and filtering layer in a reaction space.

2. Description of Related Art Including Information Disclosed Under 37 CFR 1.97 and 37 CFR 1.98.

There are currently available optical and electrochemical blood-glucose testers. But the portable blood-glucose tester along with the electrochemical sensing test block always has larger measurement error primarily owing to HCT in the blood samples. The effects caused by said HCT error include: different blood concentrations will lead to inconsistent electronic transfer rate and affect the final measurement value; inconsistent volume of test serum will further lead to differences of measurement criterion.

The sensing test block is structurally designed in a manner wherein a slotted groove is set laterally onto the sensing end of the block body, such that the blood sample is dripped into the slotted groove as per siphon principle, enabling to generate a reaction with a preset internal electrochemical reaction portion. However, when the blood sample is dripped by the user into the groove, the quantity of test serum absorbed into the groove to reach the electrochemical reaction portion may vary under the interferences of different blood corpuscle concentrations due to inconsistent percentage of serum and blood corpuscle concentrations. Said difference of blood corpuscle concentrations will certainly lead to inconsistent measurement criterions and poorer accuracy.

For this reason, the inventor has developed a new patent: “an electrochemical sensing test block capable of removing the interference of blood corpuscle” as disclosed by ROC patent No. M359695, wherein a porous separation and filtering layer is additionally set onto the chemical reaction zone in the reaction space of the sensing test block. The sensing test block is used in a manner that the blood corpuscle in the test blood sample is filtered by the porous separation and filtering layer, and serum could enter into the chemical reaction zone whilst the interference factors of HCT and oxygen content in blood can be eliminated.

Some shortcomings of the prior art are still observed from subsequent applications. That is to say although the blood corpuscle in the test blood sample could be filtered by the porous separation and filtering layer, the input of test blood sample is blocked simultaneously, so that the serum reaching the chemical reaction zone will be delayed, thus affecting the performance and quality of the sensing test block.

Thus, to overcome the aforementioned problems of the prior art, it would be an advancement if the art to provide an improved structure that can significantly improve the efficacy.

Therefore, the inventor has provided the present invention of practicability after deliberate design and evaluation based on years of experience in the production, development and design of related products.

### BRIEF SUMMARY OF THE INVENTION

Based on the unique construction of the present invention wherein the “sensing test block with rapid conductive reaction effect” allows said porous separation and filtering layer and said capillary guiding and diffusion portion to be set onto the chemical reaction layer in the reaction space, the specimen is guided into the reaction space through the specimen inlet, and then rapidly absorbed and diffused onto the porous separation and filtering layer via the capillary action of the capillary guiding and diffusion portion. Next, the serum in the specimen could be precipitated to reach the chemical reaction layer through porous separation and filtering layer, such that the detection speed of the sensing test block can be further increased to improve substantially its performance and quality.

Additionally, based on the structural configuration wherein a reinforced specimen absorber is defined on the capillary guiding and diffusion portion correspondingly to the periphery of the porous separation and filtering layer, the specimen is guided into the capillary guiding and diffusion portion, and then rapidly absorbed and diffused towards the reinforced specimen absorber, thus avoiding any accuracy interference arising from stagnation of the specimen in the center of the reaction space.

Although the invention has been explained in relation to its preferred embodiment, it is to be understood that many other possible modifications and variations can be made without departing from the spirit and scope of the invention as hereinafter claimed.

### BRIEF DESCRIPTION OF THE SEVERAL VIEWS OF THE DRAWINGS

FIG. 1 is an exploded perspective view of the preferred embodiment of the sensing test block of the present invention.

FIG. 2 is a partially assembled perspective view of the preferred embodiment of the sensing test block of the present invention.

FIG. 3 is a partial sectional view and detection status view of the preferred embodiment of the sensing test block of the present invention.

FIG. 4 is another schematic view and detection status view of the sensing test block of the present invention.

FIG. 5 is a schematic view of the present invention wherein the venting portion is set onto several locations.

FIG. 6 is a schematic view of the present invention wherein the reaction detector is of an optical type.

FIG. 7 is a view of a variation of the structure disclosed in FIG. 6.

### DETAILED DESCRIPTION OF THE INVENTION

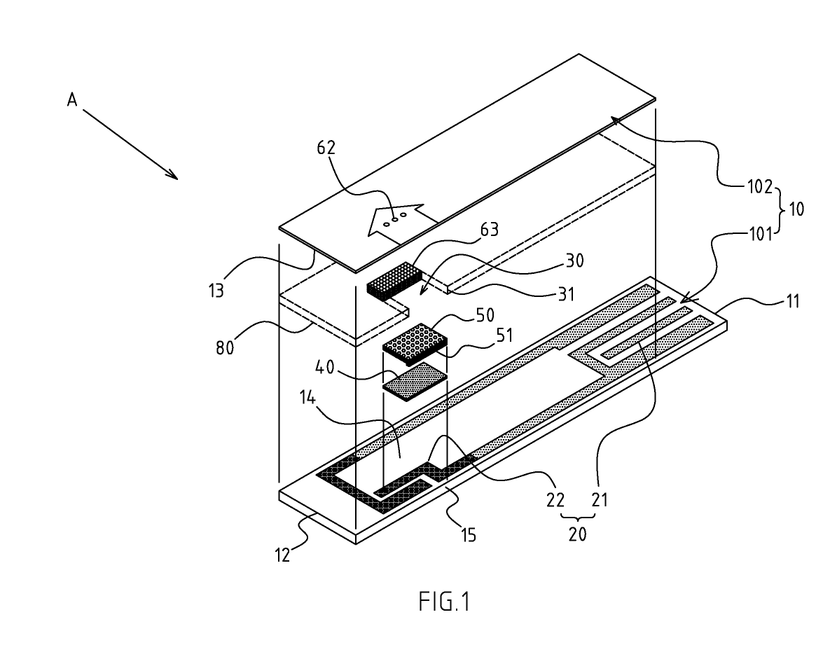

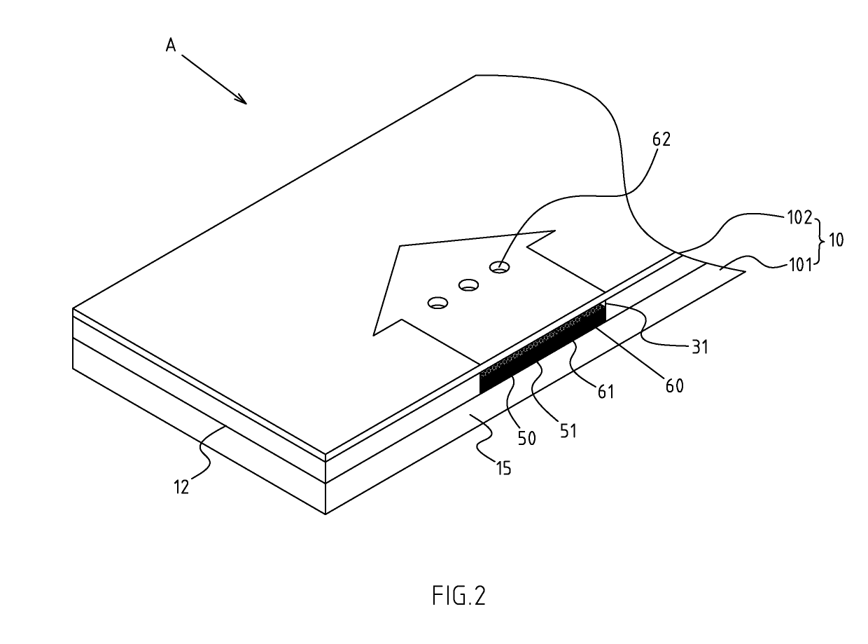

FIGS. 1, 2 and 3 depict preferred embodiments of a sensing test block of the present invention with rapid conductive reaction effect, which, however, are provided for only explanatory objective for patent claims.

The sensing test block A comprises a test block body 10 having an insertion end 11 and a sensing end 12, of which the sensing end 12 includes a top wall 13, a bottom wall 14 and a lateral portion 15.

A reaction detector 20 is arranged onto the test block body 10. The reaction detector 20 having a detection zone 21 and a reaction zone 22. The reaction detector is an electrochemical one comprised of positive and negative electrode plates (disclosed in FIG. 1). The detection zone 21 is located correspondingly to the insertion end 11 of the test block body 10, and the reaction zone 22 located correspondingly to the sensing end 12 of the test block body 10.

A reaction space 30 is set onto the sensing end 12 of the test block body 10, and located correspondingly to the reaction zone 22 of the reaction detector 20. The reaction space 30 is provided with a specimen inlet 31.

A chemical reaction layer 40 is set onto the reaction zone 22 of the reaction detector 20. The chemical reaction layer 40 made of ferment is used to generate a chemical reaction with glucose in the specimen (e.g.: test blood sample). As for the electrochemical preferred embodiment, some electrons are generated and accumulated in the reaction zone 22 of the reaction detector 20, such that when the reaction detector 20 is energized to provide a fixed voltage, the quantity of electrons accumulated in the reaction zone 22 could be detected.

A porous separation and filtering layer 50 is set into the reaction space 30 and overlapped onto the chemical reaction layer 40. The aperture of the porous separation and filtering layer 50 must be less than 6 μm, so the blood corpuscle in the test blood sample could be separated. Given the fact that the external diameter of blood corpuscle in the human bodies and animals is larger than 6 μm, the design of the aperture allows the serum to pass through while the blood corpuscle is blocked and filtered. Moreover, the porous separation and filtering layer 50 is provided with a guiding portion 51 that's mated with the specimen inlet 31 in the reaction space 30.

A capillary guiding and diffusion portion 60 is set into the reaction space 30 and overlapped onto the porous separation and filtering layer 50. The capillary guiding and diffusion portion 60 is provided with a specimen guiding portion 61 that is mated with the specimen inlet 31 in the reaction space 30. At least a venting portion 62 is set onto the capillary guiding and diffusion portion 60, and also located correspondingly to the external side of the porous separation and filtering layer 50, making the venting portion 62 farther from the specimen inlet 31 than the porous separation and filtering layer 50.

When the specimen 70 is guided from the specimen inlet 31 of the reaction space 30, the specimen 70 could be rapidly absorbed and diffused onto the porous separation and filtering layer 50 through the capillary action of the capillary guiding and diffusion portion 60 along with the venting exhaust at rear of the venting portion 62. The serum in the specimen 70 could be precipitated to reach the chemical reaction layer 40 through porous separation and filtering layer 50.

Referring to FIG. 1, the test block body 10 is fabricated by the superposition of an insulating substrate 101 (e.g.: plastic plate) and a covering plate 102. The bottom wall 14 of the sensing end 12 is formed by the insulating substrate 101, and the top wall 13 of the sensing end 12 formed by the covering plate 102. Moreover, the insulating substrate 101 and the covering plate 102 are fixed securely via an adhesion layer 80. A recession is reserved on the adhesion layer 80 correspondingly to the sensing end, so as to form the reaction space 30.

Of which, the height of the capillary guiding and diffusion portion 60 ranges from 0.1 mm to 0.2 mm for desired capillary guiding effect.

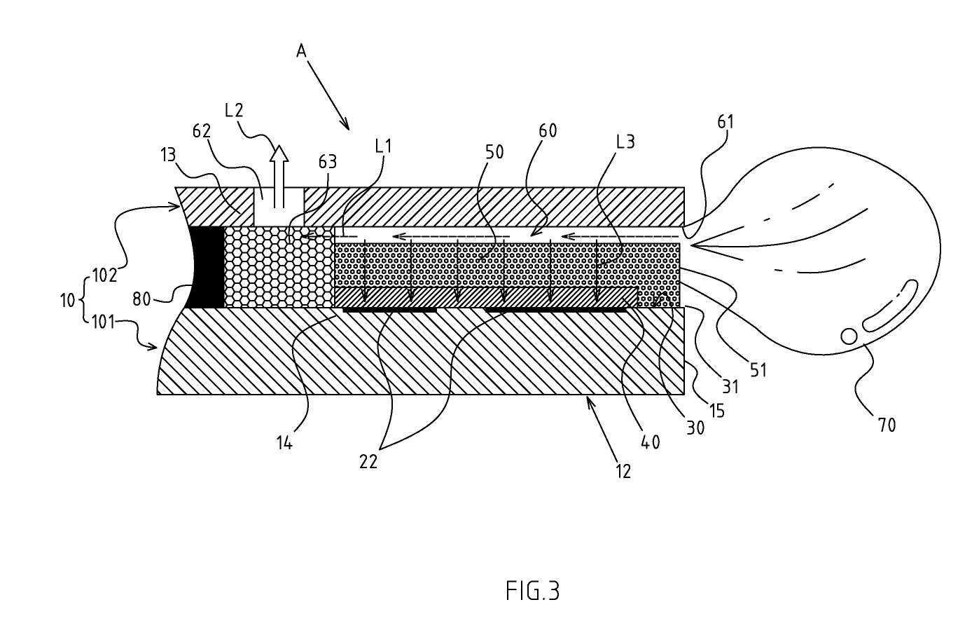

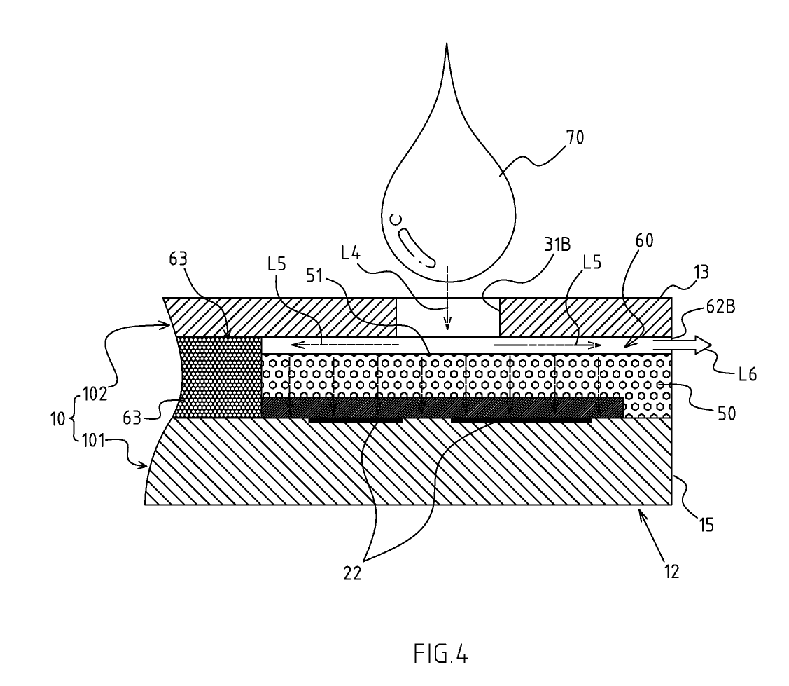

Of which, the specimen inlet 31 of the reaction space 30 is set onto either the top wall 13 or the lateral portion 15 of the sensing end 12. Referring to FIG. 3, said specimen inlet 31 is set onto the lateral portion 15 of the sensing end 12. Referring also to FIG. 4, said specimen inlet 31B is set onto the top wall 13 of the sensing end 12.

Of which, the venting portion 62 of the capillary guiding and diffusion portion 60 is set onto either the top wall 13 or the lateral portion 15 of the sensing end 12. Referring to FIG. 3, said venting portion 62 is set onto the top wall 13 of the sensing end 12. Referring also to FIG. 4, said venting portion 62B is set onto the lateral portion 15 of the sensing end 12.

Of which, the porous separation and filtering layer 50 is made of flaky bamboo charcoal, which is characterized by its capillary filtering effect. The blood corpuscle in the test blood sample is already removed after being filtered by the porous separation and filtering layer 50, so the measurement result is free from the influence of HCT in the blood.

Referring to FIGS. 1 and 3, a reinforced specimen absorber 63 (e.g.: absorbent cotton) is defined on the capillary guiding and diffusion portion 60 correspondingly to the periphery of the porous separation and filtering layer 50. Referring to FIG. 3, after the specimen 70 is guided into the uppermost capillary guiding and diffusion portion 60 in the reaction space 30 via the specimen inlet 31, the specimen 70 could be rapidly absorbed by the reinforced specimen absorber 63 and diffused towards the reinforced specimen absorber 63 (marked by arrow L1), thus avoiding any accuracy interference arising from stagnation of the specimen 70 in the center of the reaction space 30. As for the test blood sample, the whole blood retention time is shortened, and the blood could be diffused rapidly around the reaction zone, such that no stagnation of red blood corpuscle occurs in the reaction zone 22, and only serum can be infiltrated into the chemical reaction layer 40 with better test results.

Based upon above-specified structure, the sensing test block A of the present invention is used to measure the blood sugar of the human body in collaboration with a blood-glucose tester. Referring to FIG. 3, the specimen 70 (i.e.: blood) could be dripped by the user into the reaction space 30 of the sensing test block A through the specimen inlet 31. After the specimen 70 is guided into the uppermost capillary guiding and diffusion portion 60 in the reaction space 30 via the specimen inlet 31, the specimen 70 could be rapidly absorbed and diffused on the porous separation and filtering layer 50 (marked by arrow L1). While the specimen 70 is absorbed into the capillary guiding and diffusion portion 60, air could be discharged by the venting portion 62 (marked by arrow L2), such that the specimen 70 could be guided quickly without any jamming. Next, the serum in the specimen 70 could be precipitated to pass through the porous separation and filtering layer 50 (marked by arrow L3). Due to the porous structure of the porous separation and filtering layer 50, the blood corpuscle in the specimen 70 could be blocked and filtered, and only serum in the specimen 70 could reach the chemical reaction zone 50. In such case, a chemical reaction between the glucose in the specimen 70 and the chemical reaction layer 40 will generate electrons, which are accumulated in the reaction zone 22 of the reaction detector 20. Next, the reaction detector 20 of the sensing test block A is energized through the blood-glucose tester to provide a fixed voltage, so the quantity of electrons accumulated in the reaction zone 22 could be detected. The detected current is calculated by a conversion formula preset in the blood-glucose tester to obtain the concentration of glucose in the specimen 70.

Referring to FIG. 4, as the specimen inlet 31B is set onto the top wall 13 of the sensing end 12, the specimen 70 is dripped into the specimen inlet 31B (marked by arrow L4), and then diffused transversely after entering into the capillary guiding and diffusion portion 60 (marked by arrow L5). While the specimen 70 is absorbed into the capillary guiding and diffusion portion 60, air could be discharged by the venting portion 62B set on the lateral portion 15 of the sensing end 12 (marked by arrow L6).

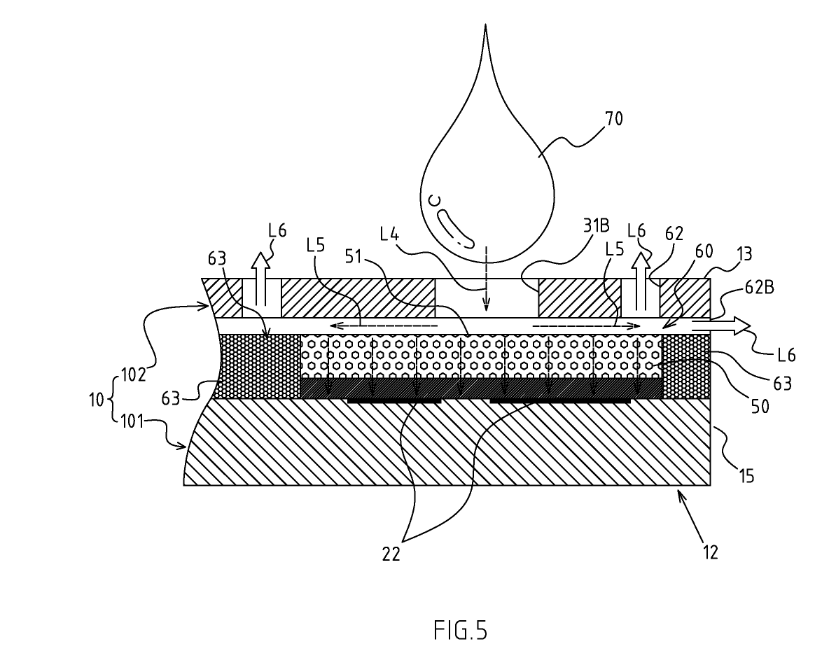

Referring to FIG. 5, the venting portions 62, 62B of the capillary guiding and diffusion portion 60 can be also set simultaneously onto the top wall 13 and lateral portion 15, helping to discharge air by multiple ways (marked by arrow L6).

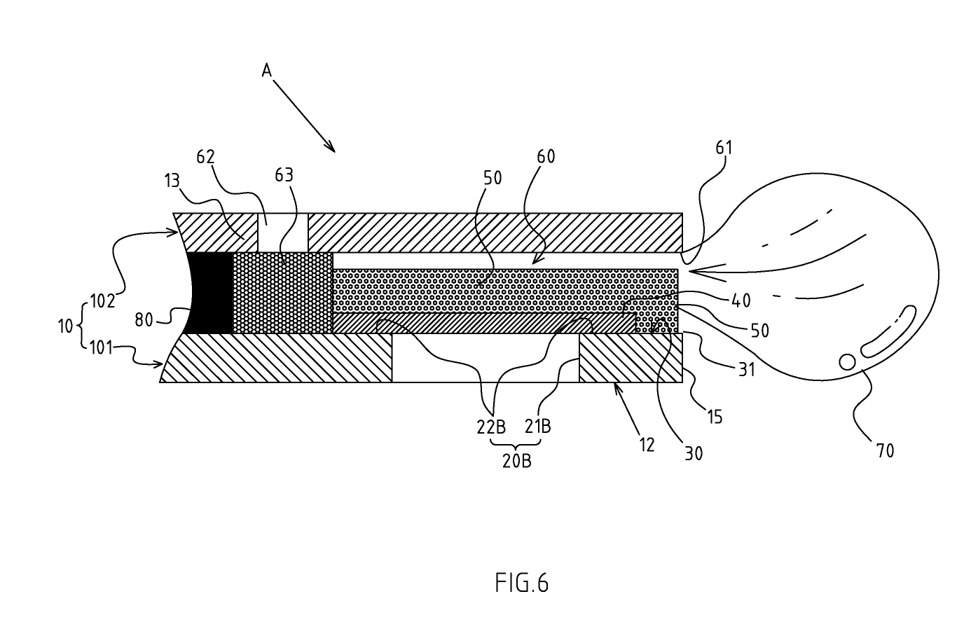

Referring to FIG. 6, the reaction detector 20B is also of an optical type (Colorimetric or Photometric or Reflectometric). Both the detection zone 21B and reaction zone 22B are set correspondingly to the sensing end 12 of the test block body 10. The detection zone 21B is set onto either the top wall or bottom wall of the sensing end 12 as a viewing window. With the help of the optical reaction detector 20B, the detection principle is implemented in a way that the specimen in the chemical reaction layer 40 could yield chemical reaction to bring about color change, enabling inspection through the detection zone 21B by human eyes or detectors.

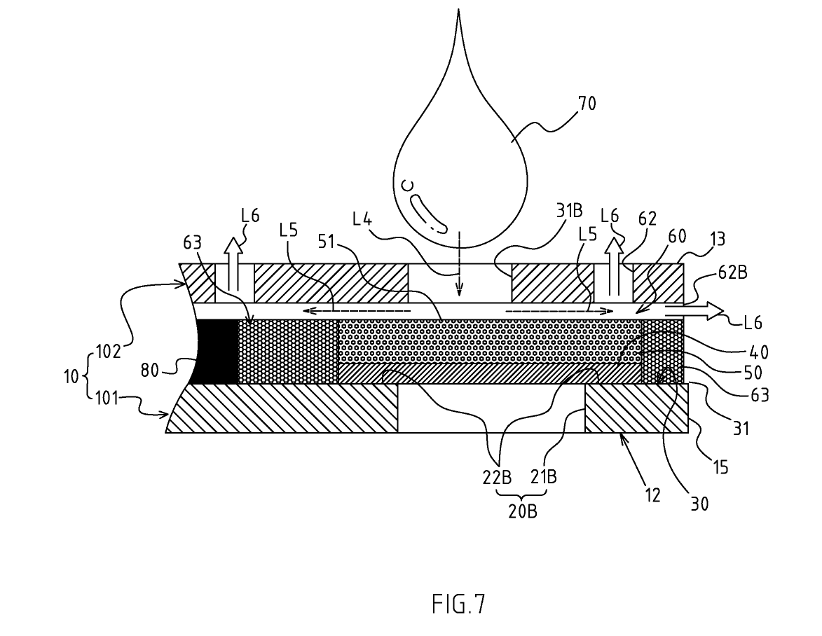

Referring also to FIG. 7, the reaction detector 20B is of an optical framework, whereby the venting portions 62, 62B of the capillary guiding and diffusion portion 60 can also be set simultaneously onto the top wall 13 and lateral portion 15.

1. A sensing test block with rapid conductive reaction effect comprising:
a test block body having an insertion end and a sensing end, of which the sensing end includes a top wall, a bottom wall and a lateral portion;
a reaction detector arranged onto the test block body; the reaction detector consists of having a detection zone and a reaction zone; the reaction zone is located correspondingly to the sensing end of the test block body;
a reaction space set onto the sensing end of the test block body, and located correspondingly to the reaction zone of the reaction detector; the reaction space is provided with a specimen inlet;
a chemical reaction layer set onto the reaction zone of the reaction detector;
a porous separation and filtering layer set into the reaction space and overlapped onto the chemical reaction layer; the aperture of the porous separation and filtering layer being less than 6 μm, so the blood corpuscle in the test blood sample could be separated; the porous separation and filtering layer is provided with a guiding portion that is mated with the specimen inlet in the reaction space;
a capillary guiding and diffusion portion; set into the reaction space and overlapped onto the porous separation and filtering layer; the capillary guiding and diffusion portion is provided with a specimen guiding portion is mated with the specimen inlet in the reaction space; the coverage of the capillary guiding and diffusion portion is larger than that of the porous separation and filtering layer; and
a venting portion set onto at least one location of the capillary guiding and diffusion portion; the venting portion is located correspondingly to the external side of the porous separation and filtering layer, making the venting portion farther from the specimen inlet than the porous separation and filtering layer;
when the specimen is guided from the specimen inlet of the reaction space, the specimen could be rapidly absorbed and diffused onto the porous separation and filtering layer through the capillary action of the capillary guiding and diffusion portion along with the venting exhaust at rear of the venting portion; and the serum in the specimen could be precipitated to reach the chemical reaction layer through porous separation and filtering layer.

2. The sensing test block defined in claim 1, wherein said test block body is fabricated by the superposition of an insulating substrate and a covering plate; the bottom wall of the sensing end is formed by the insulating substrate, and the top wall of the sensing end formed by the covering plate; the insulating substrate and the covering plate are fixed securely via an adhesion layer; a recession is reserved on the adhesion layer correspondingly to the sensing end, so as to form the reaction space.

3. The sensing test block defined in claim 1, wherein the height of the capillary guiding and diffusion portion ranges from 0.1 mm to 0.2 mm.

4. The sensing test block defined in claim 1, wherein the specimen inlet of the reaction space is set onto either the top wall or the lateral portion of the sensing end.

5. The sensing test block defined in claim 1, wherein the venting portion of the capillary guiding and diffusion portion is set onto either the top wall or the lateral portion of the sensing end.

6. The sensing test block defined in claim 1, wherein said reaction detector is an electrochemical one comprised of positive and negative electrode plates; the detection zone is located correspondingly to the insertion end of the test block body, and the reaction zone located correspondingly to the sensing end of the test block body;

7. The sensing test block defined in claim 1, wherein the reaction detector is of an optical type (Colorimetric or Photometric or Reflectometric); both the detection zone and reaction zone are set correspondingly to the sensing end of the test block body; the detection zone is set onto either the top wall or bottom wall of the sensing end as a viewing window, enabling inspection by human eyes or detectors.

8. The sensing test block defined in claim 1, wherein a reinforced specimen absorber is defined on the capillary guiding and diffusion portion correspondingly to the periphery of the porous separation and filtering layer.
